(19) United States
(12) Patent Application Publication
(10) Pub. No.: US 2004/0204962 A1
(43) Pub. Date: Oct. 14, 2004

(54) Automated patient compliancy monitoring system and method

(76) Inventors: Steven Joseph Howser, Lakewood, CA (US); Tommy Mel Simonis, West Corvina, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 10/775,729

(22) Filed: Feb. 10, 2004

Related U.S. Application Data
(60) Provisional application No. 60/468,950, filed on May 7, 2003.
(60) Provisional application No. 60/463,184, filed on Apr. 14, 2003.

(51) Int. Cl. G06F 17/60; A61B 5/00

(52) U.S. Cl. 705/2; 600/300

(57) ABSTRACT

An automatic patient compliancy monitoring method includes communicating a patient compliancy data generating device and a charging and communication station; transmitting patient compliancy data to the charging and communication station; charging the patient compliancy data generating device with the charging and communication station; communicating the charging and communication station and a central server; transmitting patient compliancy data from the charging and communication station to a central server; communicating the central server and a clinician computer; transmitting patient compliancy data from the central server to a clinician computer; and generating a patient use report from the patient compliancy data.

### CROSS-REFERENCE TO RELATED APPLICATIONS

This application claims the benefit of prior provisional application 60/468,950, filed May 7, 2003, and prior provisional application 60/463,184, filed Apr. 14, 2003, under 35 U.S.C. 119(e).

### FIELD OF THE INVENTION

The present invention relates, in general, to systems and methods that provide an internet-based automated patient compliancy monitoring and automated patient use report generation, and, in particular, to medical transcutaneous stimulator systems and methods that provide internet-based automated patient compliancy monitoring and automated patient use report generation.

### BACKGROUND OF THE INVENTION

Medical stimulators that provide electrical stimulation signals to a patient are used to provide short and long term pain relief through transcutaneous electrical nerve stimulation (“TENS”) and to stimulate and rehabilitate muscles through neuromuscular stimulation (“NMS” or “EMS”). As used herein, a “transcutaneous electrical stimulator” includes at least a TENS stimulator, a NMS or EMS stimulator, or any other medical stimulator used to transcutaneously deliver therapeutic electrical impulses. These types of medical stimulators typically include lead wires with distal electrodes that are attached to the patient's skin. The transcutaneous electrical stimulator sends electrical stimulation signals into the muscles and nerves though the attached electrodes. The electrical stimulation signals produced by the transcutaneous electrical stimulator are in the form of a train of electrical pulses which may be modulated in rate and/or intensity.

A number of existing transcutaneous electrical stimulators generate compliancy data related to the performance of the transcutaneous electrical stimulator. Compliancy data is patient use data related to the performance of the transcutaneous medical stimulator during use of the transcutaneous medical stimulator by a patient. It is important for the prescribing clinician to obtain a compliance report or patient use report so that the clinician knows the patient is using the transcutaneous medical stimulator and the transcutaneous medical stimulator is working as prescribed. The patient use report may also be important for other purposes such as medical billing or reimbursement.

Currently, one company has incorporated the use of a removable data module (i.e., a compact flash card) as a way to collect compliancy data on the transcutaneous medical stimulator. The compact flash card may be inserted into and removed from a slot in the transcutaneous medical stimulator. An equipment distributing company that distributes the transcutaneous medical stimulator relies on the patient to remove the compact flash card from the transcutaneous medical stimulator after use, and mail the compact flash card to the equipment distributing company. The equipment distributing company then generates a compliance report, and sends the compliance report to the prescribing clinician.

Problems with this process that either delay or prevent the collection of the compliance data and generation of the compliancy report include it relies on the patient sending the compact flash card to the equipment distributing company (if the patient forgets or decides not to send the compact flash card to the equipment distributing company, a compliance report will never be generated), the compact flash card may become lost in the mail or damaged during transit (resulting in compliancy data being lost and a compliance report never being generated), and the compact flash card may become lost at the equipment distributing company (resulting in compliancy data being lost and a compliance report never being generated). With this process, the time it takes for a compliance report to be generated depends on factors such as the level of mail service from the patient to the equipment distributor, the amount of time it takes to reading data and a generate a compliancy report at the equipment distributor, and the mail service from distributor to clinician.

The present invention solves these problems with past methods of reporting compliancy data of a transcutaneous electrical stimulator to a prescribing clinician by automating the compliancy data reporting process and using the internet to securely transfer compliancy data to the prescribing clinician.

### SUMMARY OF THE INVENTION

Accordingly, an aspect of the invention involves an automatic patient compliancy monitoring method. The method includes communicating a patient compliancy data generating device and a charging and communication station; transmitting patient compliancy data to the charging and communication station; charging the patient compliancy data generating device with the charging and communication station; communicating the charging and communication station and a central server; transmitting patient compliancy data from the charging and communication station to a central server; communicating the central server and a clinician computer; transmitting patient compliancy data from the central server to a clinician computer; and generating a patient use report from the patient compliancy data.

Another aspect of the invention involves automatic patient compliancy monitoring system. The automatic patient compliancy monitoring system includes a patient compliancy data generating device for use with a patient, the patient compliancy data generating device configured to generate and store patient compliancy data; and a charging and communication station configured to receive transmitted patient compliancy data from the patient compliancy data generating device, charge the patient compliancy data generating device, and communicate with a central server and transmit patient compliancy data thereto for reporting to a clinician computer for patient compliance verification.

A further aspect of the invention involves an automatic patient compliancy monitoring method including using a patient compliancy data generating device to provide a medically therapeutic benefit to a patient; docking the patient compliancy data generating device with a charging and communication station after one or more therapeutic treatments with the patient compliancy data generating device; charging the patient compliancy data generating device with the charging and communication station; transmitting patient compliancy data from the patient compliancy data generating device to the charging and communication station; and transmitting the patient compliancy data from the charging and communication station to a clinician computer over the internet for verifying proper use of the patient compliancy data generating device by a patient.

### BRIEF DESCRIPTION OF THE DRAWINGS

FIG. 1 is a schematic diagram of an embodiment of a an automated compliancy data reporting system for a transcutaneous electrical stimulator.

FIG. 2 is a flow chart of an exemplary method of using the automated compliance data reporting system for automatically reporting compliancy data of a transcutaneous electrical stimulator.

FIG. 3 is a block diagram illustrating an exemplary computer as may be used in connection with the embodiments described herein.

### DETAILED DESCRIPTION OF PREFERRED EMBODIMENTS

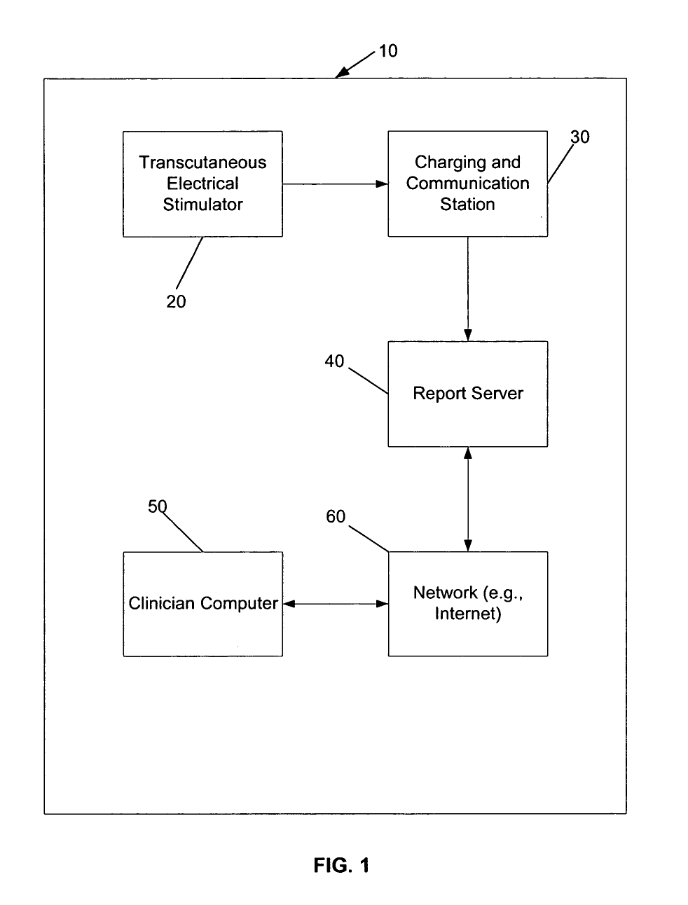

With reference to FIG. 1, an embodiment of an automated compliancy data reporting system 10 for a transcutaneous electrical stimulator 20 will now be described. The compliancy data reporting system 10 includes the transcutaneous electrical stimulator 20, a combination charging and communication (interfacing) station 30 and a report server 40. The compliancy data reporting system 10 enables compliancy data from the transcutaneous electrical stimulator 20 to be accessible to or reported to a clinician or clinician computer 50 over a computer network such as the internet 60. Examples of compliancy data may include patient use data such as, but not by way of limitation, amplitude settings, time used, date used, total treatment time, and/or mode of treatment.

It should be noted, although the automated compliancy data reporting system 10 and method will be described in conjunction with a transcutaneous electrical stimulator 20, in alternative embodiments, the device 20 may be a medical device for a patient other than a transcutaneous electrical stimulator. The device 20 generates data related to performance of the device 20 and/or patient data for determining patent compliance.

The transcutaneous electrical stimulator 20 is preferably used externally to provide electrical stimulation signals to a patient transcutaneously, through the patient's skin. The electrical stimulation signals may be used to provide short and long term pain relief through transcutaneous electrical nerve stimulation (“TENS”), to stimulate and rehabilitate muscles through neuromuscular stimulation (“NMS”), provide interferential therapy, and/or provide electrical stimulation for edema reduction.

The transcutaneous electrical stimulator 20 preferably operates on one or more rechargeable batteries or one or more other rechargeable or renewable power sources. The transcutaneous electrical stimulator 20 may include numerous input buttons or a touch keypad and a digital readout for controlling the transcutaneous electrical stimulator 20. The transcutaneous electrical stimulator 20 may include an appropriate connector (e.g., a multi-pin connector, a cable) for electrically coupling the transcutaneous electrical stimulator 20 to the combination charging and communication station 30 for communicating compliancy data between the transcutaneous electrical stimulator 20 and the combination charging and communication station 30, and for recharging the one or more rechargeable batteries of the transcutaneous electrical stimulator 20 via the combination charging and communication station 30. An AC adapter may be used to charge the rechargeable batteries or separately powering the transcutaneous electrical stimulator 20. The transcutaneous electrical stimulator 20 may include a countdown timer with automatic shut-off. The transcutaneous electrical stimulator 20 may also include a compliance meter.

Preferably, the transcutaneous electrical stimulator 20 provides the following three types of therapies: 1) TENS, 2) interferential, and 3) neuromuscular stimulation. The transcutaneous electrical stimulator 20 may include five pre-set protocols or modalities. The pre-set protocols may include the following: 1) STD (Standard/Interferential) with a 20 minute treatment program where 10 minutes are set at 1-10 Hz and 10 minutes are set at 80-150 Hz; 2) FSB (Full Sweep/Interferential) with a 30 minute treatment program where frequency is ramped, is in the range of 1-150 Hz, and includes 6 second sweep time; 3) BCK (Edema) with a 60 minute treatment program where frequency is ramped, is in the range of 1-10 Hz, and includes 6 second sweep time; 4) CNT (TENS) with a 60 minute treatment program where frequency is set at 100 Hz; and 5) STM (Muscle Stimulator) with a 60 minute treatment program where frequency is set at 48 Hz, and the therapy is provided in 6 second on/6 second off intervals. The pre-set protocols may include three programmable clinician set protocols M1, M2, M3, where memory functions 1-3 allow a clinician to place different protocols into memory for different individuals. In an alternative embodiment, protocols different than the ones described above may be performed with the transcutaneous electrical stimulator 20.

The transcutaneous electrical stimulator 20 includes firmware and/or software to perform the aforementioned treatment therapies and to generate and store compliancy data. One or more patient-approved, self-adhesive conductive electrodes may be connected to the transcutaneous electrical stimulator 20 through one or more connection wires and affixed to the patient's skin for delivering electrical stimulation waveform signals into the muscles and nerves though the patient's skin.

The combination charging and communication station 30 may be a cradle or other interfacing device for connecting the transcutaneous electrical stimulator 20 for communication and charging functions. A charging transformer may be provided with the combination charging and communication station 30 for charging the rechargeable batteries of the transcutaneous electrical stimulator 20. The combination charging and communication station 30 may include a modem (e.g., a phone modem) to connect the station 30 to a main server/web server. The station 30 may include an appropriate connector for electrically coupling the transcutaneous electrical stimulator 20 to the station 30 for communicating compliancy data between the transcutaneous electrical stimulator 20 and the station 30, and for recharging the one or more rechargeable batteries of the transcutaneous electrical stimulator 20. The station 30 includes appropriate firmware and/or software to perform the functions and steps described herein.

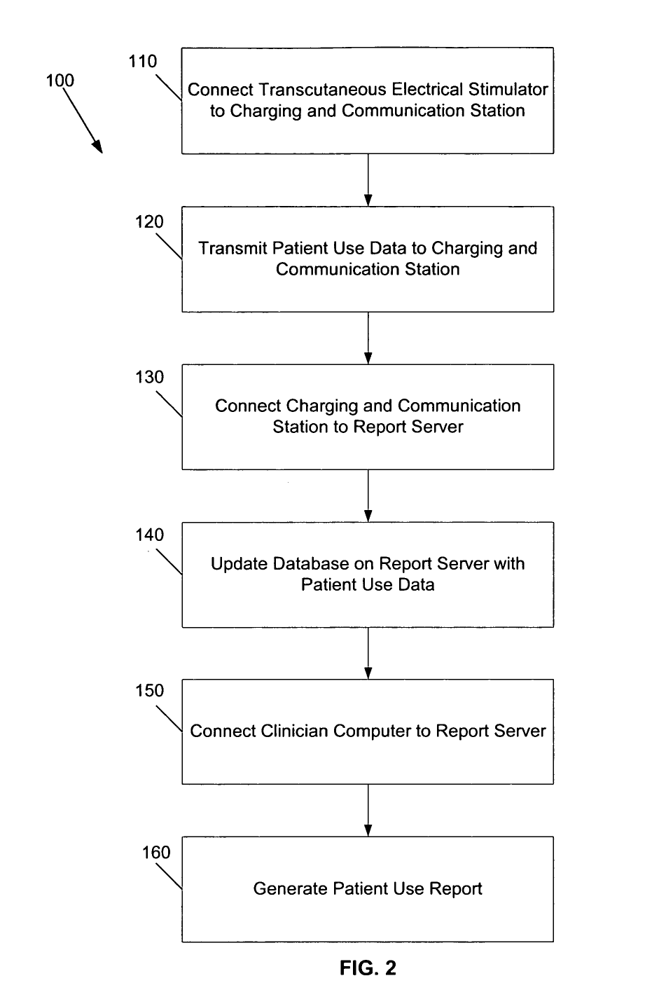

With reference to FIG. 2, an exemplary method 100 of using the compliance data reporting system for automatically reporting compliancy data of the transcutaneous electrical stimulator 20 will now be described. As discussed above, in alternative embodiments, the method 100 may be a method of using the compliance data reporting system for automatically reporting compliancy data of a medical device for a patient other than a transcutaneous electrical stimulator. In such a method, the device 20 may be any medical device for a patient that generates data related to performance of the device 20 and/or patient data for determining patent compliance.

As the patient uses the transcutaneous electrical stimulator 20, patient data or user information (e.g., amplitude settings, time used, date used, total treatment time, mode of treatment) is generated and stored in the transcutaneous electrical stimulator 20. After each treatment or multiple treatments, at step 110, the patient connects or docks the transcutaneous electrical stimulator 20 to the combination charging and communication station 30 for charging the rechargeable batteries of the transcutaneous electrical stimulator 20. It is important to use one or more rechargeable batteries or other power sources that require recharging or renewing in the transcutaneous electrical stimulator 20 to make sure the patient connects the transcutaneous electrical stimulator 20 to the station 30 after each treatment or multiple treatments so that the patient use data can be transmitted to the report server 40.

At step 120, when the transcutaneous electrical stimulator 20 is connected or docked to the station 30, compliancy data (i.e., patient user log, patient use report, patient use data, compliance menu) from the transcutaneous electrical stimulator 20 is automatically downloaded to the station 30, where it is stored in non-volatile memory. At the same time or a different time, the one or more rechargeable batteries of the transcutaneous electrical stimulator 20 may be charged by the station 30.

At step 130, at a designated time, the station 30 automatically connects, via the internal modem, to the report server 40 (directly or through one or more servers) and uploads all of the patient use data to the report server 40. The connection and transmission preferably occurs at a designated time, during off-peak hours over the internet. The patient use data may include a device number for the transcutaneous electrical stimulator 20 for identifying the transmitted patient use data.

At step 140, the report server 40 automatically updates an information database specific to the transcutaneous electrical stimulator 20 with the new patient use data. In another embodiment of the invention, the report server 40 may automatically send a communication (e.g., email) to the clinician's computer 50 notifying the clinician of the new patient use data added to the report server 40 (or include the new compliancy report or patient use report in the communication). The communication occurs automatically on a periodic basis (e.g., daily, weekly, bi-weekly, monthly, bimonthly).

At step 150, the clinician or clinician computer 50 connects to the report server 40 via a clinician computer and the internet 60. The clinician may use the clinician computer 50 to navigate to the report server 40 using a website address for the report server 40. The clinician computer 50 may be used by the clinician to log onto the report server 40. Preferably, the clinician must enter a secure access name and a password, which are verified, by the report server 40 for security purposes. Once on the report server 40, the clinician accesses the information database and retrieves the patient use data (i.e., patient use log) specific to the patient/transcutaneous electrical stimulator 20.

At step 160, a patient use report for the patient is generated by the report server 40 and downloaded to the clinician computer 50. The patient user report may be printed for the clinician's records and/or transmitted to a proper authority for billing, reimbursement, or other purposes (e.g. submittal to a work comp carrier and/or insurance company).

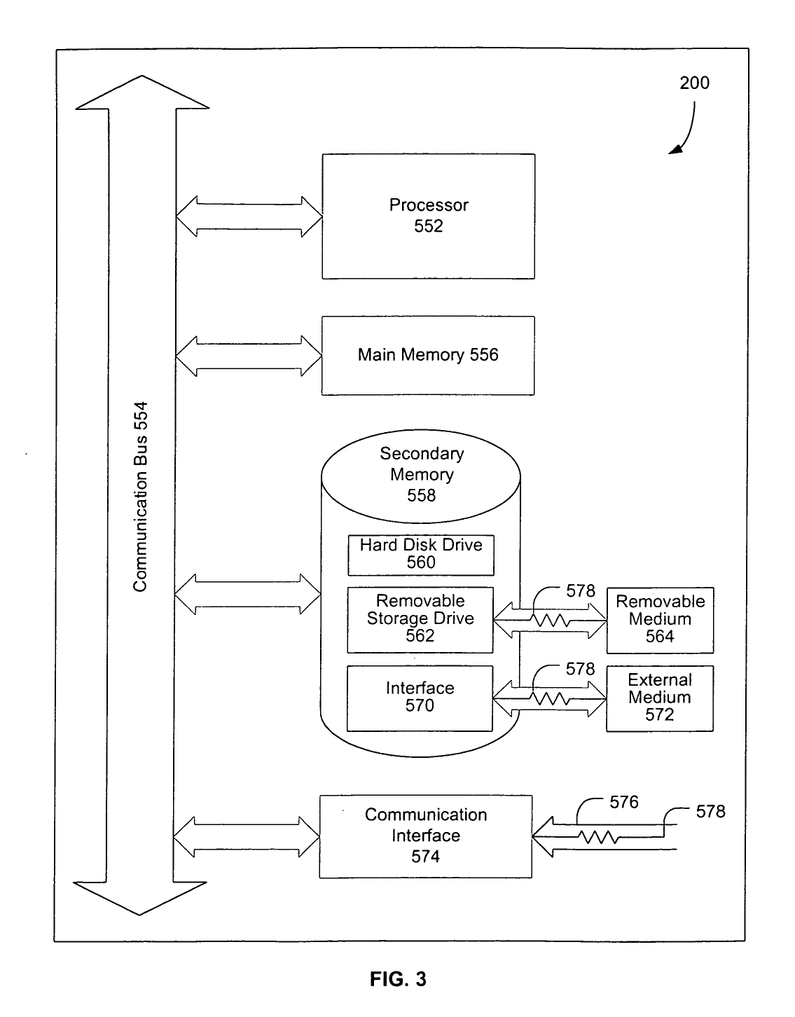

FIG. 3 is a block diagram illustrating an exemplary computer 200 as may be used in connection with the embodiments described herein. For example, the computer 200 may be used in conjunction with receiving, processing, storing, and transmitting data as described above with respect to the report server 40. However, other computers and/or architectures may be used, as will be clear to those skilled in the art. Further, the description of many of the elements of the computer 200 described is applicable to the transcutaneous electrical stimulator 20 (or other medical device 20), the combination charging and communication station 30, a computer of the clinician, and one or more possible servers in addition to the report server 40 as described above.

The computer 200 preferably includes one or more processors, such as processor 552. Additional processors may be provided, such as an auxiliary processor to manage input/output, an auxiliary processor to perform floating point mathematical operations, a special-purpose microprocessor having an architecture suitable for fast execution of signal processing algorithms (e.g., digital signal processor), a slave processor subordinate to the main processing system (e.g., back-end processor), an additional microprocessor or controller for dual or multiple processor systems, or a coprocessor. Such auxiliary processors may be discrete processors or may be integrated with the processor 552.

The processor 552 is preferably connected to a communication bus 554. The communication bus 554 may include a data channel for facilitating information transfer between storage and other peripheral components of the computer 200. The communication bus 554 further may provide a set of signals used for communication with the processor 552, including a data bus, address bus, and control bus (not shown). The communication bus 554 may comprise any standard or non-standard bus architecture such as, for example, bus architectures compliant with industry standard architecture (“ISA”), extended industry standard architecture (“EISA”), Micro Channel Architecture (“MCA”), peripheral component interconnect (“PCI”) local bus, or standards promulgated by the Institute of Electrical and Electronics Engineers (“IEEE”) including IEEE 488 general-purpose interface bus (“GPIB”), IEEE 696/S-100, and the like.

Computer 200 preferably includes a main memory 556 and may also include a secondary memory 558. The main memory 556 provides storage of instructions and data for programs executing on the processor 552. The main memory 556 is typically semiconductor-based memory such as dynamic random access memory (“DRAM”) and/or static random access memory (“SRAM”). Other semiconductor-based memory types include, for example, synchronous dynamic random access memory (“SDRAM”), Rambus dynamic random access memory (“RDRAM”), ferroelectric random access memory (“FRAM”), and the like, including read only memory (“ROM”).

The secondary memory 558 may optionally include a hard disk drive 560 and/or a removable storage drive 562, for example a floppy disk drive, a magnetic tape drive, a compact disc (“CD”) drive, a digital versatile disc (“DVD”) drive, etc. The removable storage drive 562 reads from and/or writes to a removable storage medium 564 in a well-known manner. Removable storage medium 564 may be, for example, a floppy disk, magnetic tape, CD, DVD, etc.

The removable storage medium 564 is preferably a computer readable medium having stored thereon computer executable code (i.e., software) and/or data. The computer software or data stored on the removable storage medium 564 is read into the computer 200 as electrical communication signals 578.

In alternative embodiments, secondary memory 558 may include other similar means for allowing computer programs or other data or instructions to be loaded into the computer 200. Such means may include, for example, an external storage medium 572 and an interface 570. Examples of external storage medium 572 may include an external hard disk drive or an external optical drive, or and external magneto-optical drive.

Other examples of secondary memory 558 may include semiconductor-based memory such as programmable read-only memory (“PROM”), erasable programmable read-only memory (“EPROM”), electrically erasable read-only memory (“EEPROM”), or flash memory (block oriented memory similar to EEPROM). Also included are any other removable storage units 572 and interfaces 570, which allow software and data to be transferred from the removable storage unit 572 to the computer 200.

Computer 200 may also include a communication interface 574. The communication interface 574 allows software and data to be transferred between computer 200 and external devices (e.g. printers), networks, or information sources. For example, computer software or executable code may be transferred to computer 200 from a network server via communication interface 574. Examples of communication interface 574 include a modem, a network interface card (“NIC”), a communications port, a PCMCIA slot and card, an infrared interface, and an IEEE 1394 fire-wire, just to name a few.

Communication interface 574 preferably implements industry promulgated protocol standards, such as Ethernet IEEE 802 standards, Fiber Channel, digital subscriber line (“DSL”), asynchronous digital subscriber line (“ADSL”), frame relay, asynchronous transfer mode (“ATM”), integrated digital services network (“ISDN”), personal communications services (“PCS”), transmission control protocol/internet protocol (“TCP/IP”), serial line internet protocol/point to point protocol (“SLIP/PPP”), and so on, but may also implement customized or non-standard interface protocols as well.

Software and data transferred via communication interface 574 are generally in the form of electrical communication signals 578. These signals 578 are preferably provided to communication interface 574 via a communication channel 576. Communication channel 576 carries signals 578 and can be implemented using a variety of communication means including wire or cable, fiber optics, conventional phone line, cellular phone link, radio frequency (RF) link, or infrared link, just to name a few.

Computer executable code (i.e., computer programs or software) is stored in the main memory 556 and/or the secondary memory 558. Computer programs can also be received via communication interface 574 and stored in the main memory 556 and/or the secondary memory 558. Such computer programs, when executed, enable the computer 200 to perform the various functions of the present invention as previously described.

In this description, the term “computer readable medium” is used to refer to any media used to provide computer executable code (e.g., software and computer programs) to the computer 200. Examples of these media include main memory 556, secondary memory 558 (including hard disk drive 560, removable storage medium 564, and external storage medium 572), and any peripheral device communicatively coupled with communication interface 574 (including a network information server or other network device). These computer readable mediums are means for providing executable code, programming instructions, and software to the computer 200.

In an embodiment that is implemented using software, the software may be stored on a computer readable medium and loaded into computer 200 by way of removable storage drive 562, interface 570, or communication interface 574. In such an embodiment, the software is loaded into the computer 200 in the form of electrical communication signals 578. The software, when executed by the processor 552, preferably causes the processor 552 to perform the inventive features and functions previously described herein.

Various embodiments may also be implemented primarily in hardware using, for example, components such as application specific integrated circuits (“ASICs”), or field programmable gate arrays (“FPGAs”). Implementation of a hardware state machine capable of performing the functions described herein will also be apparent to those skilled in the relevant art. Various embodiments may also be implemented using a combination of both hardware and software.

It will be readily apparent to those skilled in the art that still further changes and modifications in the actual concepts described herein can readily be made without departing from the spirit and scope of the invention as defined by the following claims.

What is claimed is:

1. An automatic patient compliancy monitoring method, comprising:
communicating a patient compliancy data generating device and a charging and communication station;
transmitting patient compliancy data to the charging and communication station;
charging the patient compliancy data generating device with the charging and communication station;
communicating the charging and communication station and a central server;
transmitting patient compliancy data from the charging and communication station to a central server;
communicating the central server and a clinician computer;
transmitting patient compliancy data from the central server to a clinician computer;
generating a patient use report from the patient compliancy data.

2. The method of claim 1, wherein the patient compliancy data generating device is a medically therapeutic device that provides a therapeutic benefit to a patient, and communicating the patient compliancy data generating device and the charging and communication station includes communicating the patient compliancy data generating device and the charging and communication station after one or more therapy treatments on the patient with the patient compliancy data generating device and transmitting patient compliancy data to the charging and communication station includes automatically downloading patient compliancy data to the charging and communication station.

3. The method of claim 1, wherein charging the patient compliancy data generating device with the charging and communication station occurs simultaneously with transmitting patient compliancy data to the charging and communication station.

4. The method of claim 2, wherein communicating the charging and communication station and the central server and transmitting patient compliancy data from the charging and communication station to the central server occurs automatically at an off-peak, designated time.

5. The method of claim 1, wherein the patient compliancy data is represented by a unique identifier specific to the patient compliancy data generating device.

6. The method of claim 1, wherein the central server includes an information database specific to the patient compliancy data generating device, and the information database is automatically updated with the patient compliancy data.

7. The method of claim 1, wherein transmitting patient compliancy data from the central server to a clinician computer includes automatically sending an electronic mail communication to the clinician computer with the patient compliancy data.

8. The method of claim 1, wherein communicating the central server and a clinician computer includes communicating to the central server over the internet with the clinician computer.

9. The method of claim 1, wherein generating a patient use report from the patient compliancy data includes generating a patient use report at the central server and transmitting the report to the clinician computer.

10. An automatic patient compliancy monitoring system, comprising:
a patient compliancy data generating device for use with a patient, the patient compliancy data generating device configured to generate and store patient compliancy data; and
a charging and communication station configured to receive transmitted patient compliancy data from the patient compliancy data generating device, charge the patient compliancy data generating device, and communicate with a central server and transmit patient compliancy data thereto for reporting to a clinician computer for patient compliance verification.

11. The automatic patient compliancy monitoring system of claim 10, wherein the patient compliancy data generating device is a medically therapeutic device configured to provide a therapeutic benefit to a patient.

12. The automatic patient compliancy monitoring system of claim 10, wherein the patient compliancy data generating device and the charging and communication station are configured for automatic downloading of patient compliancy data to the charging and communication station.

13. The automatic patient compliancy monitoring system of claim 10, wherein the patient compliancy data generating device and the charging and communication station are configured for charging of the patient compliancy data generating device by the charging and communication station with simultaneous transmission of patient compliancy data to the charging and communication station.

14. The automatic patient compliancy monitoring system of claim 10, wherein the charging and communication station is configured to automatically transmit patient compliancy data to the central server occurs at an off-peak, designated time.

15. The automatic patient compliancy monitoring system of claim 10, further including a central server including an information database specific to the patient compliancy data generating device, the information database storing the patient compliancy data.

16. The automatic patient compliancy monitoring system of claim 15, wherein the central server is configured to automatically send an electronic mail communication to the clinician computer with the patient compliancy data.

17. The automatic patient compliancy monitoring system of claim 10, wherein the central server is configured to be communicated to by a clinician computer over the internet.

18. The automatic patient compliancy monitoring system of claim 10, wherein the central server is configured to generate a patient use report for transmission to the clinician computer.

19. An automatic patient compliancy monitoring method, comprising:
using a patient compliancy data generating device to provide a medically therapeutic benefit to a patient;
docking the patient compliancy data generating device with a charging and communication station after one or more therapeutic treatments with the patient compliancy data generating device;
charging the patient compliancy data generating device with the charging and communication station;
transmitting patient compliancy data from the patient compliancy data generating device to the charging and communication station;
transmitting the patient compliancy data from the charging and communication station to a clinician computer over the internet for verifying proper use of the patient compliancy data generating device by a patient.
